(19) United States
(12) Patent Application Publication
(10) Pub. No.: US 2025/0249006 A1
(43) Pub. Date: Aug. 7, 2025

(54) TREATMENT

(71) Applicant: Verona Pharma PLC, Cardiff (GB)

(72) Inventor: Tara Renae RHEAULT, Cary, NC (US)

(73) Assignee: Verona Pharma PLC, Cardiff (GB)

(21) Appl. No.: 19/044,950

(22) Filed: Feb. 4, 2025

Related U.S. Application Data
(60) Provisional application No. 63/549,608, filed on Feb. 5, 2024.

(51) Int. Cl. A61K 31/519; A61K 9/00; A61K 45/06; A61K 47/02; A61K 47/26

(52) U.S. Cl. CPC A61K 31/519; A61K 9/0078; A61K 45/06; A61K 47/02; A61K 47/26

(57) ABSTRACT

The present invention relates to a compound for use in a method of treating chronic obstructive pulmonary disease (COPD) in a patient suffering from COPD, which compound is ensifentrine or a pharmaceutically acceptable salt thereof, wherein: the patient is resistant to improvements in lung function; and treating COPD comprises (i) improving the symptoms of COPD in the patient and/or (ii) improving the quality of life of the patient. In some aspects, the compound is in an inhalable pharmaceutical composition.

### CROSS REFERENCE

This application claims the benefit of U.S. Provisional Application No. 63/549,608, filed Feb. 5, 2024, which is incorporated herein by reference in its entirety.

### FIELD OF THE INVENTION

The present invention relates to improving symptoms and/or quality of life in COPD patients with ensifentrine or a pharmaceutically acceptable salt thereof, which patients are those demonstrating a lower FEV₁ response following treatment for COPD.

### BACKGROUND OF THE INVENTION

Ensifentrine (N-(2-{(2E)-9,10-dimethoxy-4-oxo-2-[(2,4,6-trimethylphenyl)imino]-6,7-dihydro-2H-pyrimido[6,1-a]isoquinolin-3(4H)-yl}ethyl)urea; also known as RPL554) is a dual PDE3/PDE4 inhibitor and is described in WO 00/58308 A1.

As a combined PDE3/PDE4 inhibitor, ensifentrine has both bronchodilatory and anti-inflammatory activity and is useful in the treatment of respiratory disorders including chronic obstructive pulmonary disease (COPD). The chemical structure of ensifentrine is shown below.

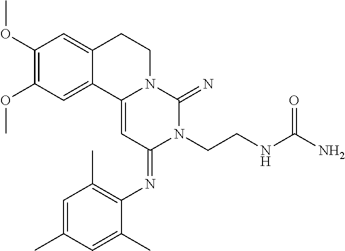 [SMILES: COc1cc2c(cc1OC)-c1c/c(=N\c3c(C)cc(C)cc3C)n(CCNC(N)=O)c(=N)n1CC2]

There are a number of approaches to treating COPD. Some approaches seek to improve lung function, for instance as measured by an increase in forced expiratory volume in 1 second (FEV₁). Other approaches focus on the symptoms of COPD, for instance by reducing symptoms or improving quality of life.

Different patients may react differently to a particular treatment for COPD, with some patients being more resistant to increases in lung function. For instance, some patients are more resistant to improvements in lung function following administration of a particular COPD treatment than other patients, with lower increases in FEV₁ observed.

Lower responses in terms of lung function in a patient are often associated with less of an improvement in symptoms and/or quality of life in the patient. This means that patients who experience a smaller improvement in lung function compared to other patients also typically experience a smaller improvement in symptoms and quality of life, therefore leading to an overall less effective treatment of COPD. It is desirable therefore to find treatments for COPD which are able to improve symptoms and/or quality of life in COPD patients, even in those patients which have a limited response in terms of lung function.

### SUMMARY OF THE INVENTION

It is a surprising finding of the present invention that ensifentrine is able to improve symptoms and/or quality of life in patients who are resistant to improvements in lung function, i.e. patients showing a relatively small improvement in lung function following treatment for COPD. Meaningful positive effects on COPD symptoms and quality of life can be achieved with ensifentrine in patients who do not necessarily show strong improvements in lung function (as measured by changes in FEV₁) following treatment.

The invention accordingly provides a compound for use in a method of treating chronic obstructive pulmonary disease (COPD) in a patient suffering from COPD, which compound is ensifentrine or a pharmaceutically acceptable salt thereof, wherein: the patient is resistant to improvements in lung function; and treating COPD comprises (i) improving the symptoms of COPD in the patient and/or (ii) improving the quality of life of the patient. Typically, treating COPD comprises improving the symptoms of COPD in the patient.

Also provided by the invention is a method for treating chronic obstructive pulmonary disease (COPD) in a patient suffering from COPD, wherein: the method comprises administering a therapeutically effective amount of a compound which is ensifentrine or a pharmaceutically acceptable salt thereof to the patient; the patient is resistant to improvements in lung function; and treating COPD comprises (i) improving the symptoms of COPD in the patient and/or (ii) improving the quality of life of the patient.

Further provided by the invention is use of a compound in the manufacture of a medicament for a method of treating chronic obstructive pulmonary disease (COPD) in a patient suffering from COPD, which compound is ensifentrine or a pharmaceutically acceptable salt thereof, wherein: the patient is resistant to improvements in lung function; and treating COPD comprises (i) improving the symptoms of COPD in the patient and/or (ii) improving the quality of life of the patient.

### DETAILED DESCRIPTION OF THE INVENTION

The patient is resistant to improvements in lung function. As such, the patient less readily experiences an improvement in lung function following a treatment to improve lung function, for instance a treatment for COPD. The patient may for instance experience a lower improvement than average in lung function following inhalation of a bronchodilator and/or an anti-inflammatory. Lung function includes factors such as the amount of air the lungs can hold and the force with which air can be expelled from the lungs. Lung function is typically assessed by spirometry and key measures include forced expiratory volume in one second (FEV₁), and in particular FEV₁ area under the curve from 0-12 h (AUC₀₋₁₂) and morning trough FEV₁.

It has been found that ensifentrine or a pharmaceutically acceptable salt thereof is able to improve COPD symptoms and quality of life (QoL) in COPD, even in patients who show a limited response in terms of improvements in lung function following a reference COPD treatment (i.e. in patients resistant to improvements in lung function). As used herein, the “reference COPD treatment” may be any treatment for COPD, for instance any treatment for COPD which would enable an assessment of the response of the lung function of the patient to the treatment of COPD. The patient may or may not have undergone the reference COPD treatment. For instance, the patient may be a patient susceptible to demonstrating a low FEV₁ response relative to baseline in the event that they were administered the reference COPD treatment. As discussed further below, the reference COPD treatment may be treatment with the compound (i.e. ensifentrine or a pharmaceutically acceptable salt thereof) or may be treatment with a different COPD treatment (for instance a known bronchodilator).

The patient resistant to improvements in lung function is typically a patient who demonstrates a low FEV₁ response relative to baseline following a reference COPD treatment. References to a patient who demonstrates a low FEV₁ response relative to baseline following a reference COPD treatment includes a patient who is susceptible to demonstrating a low FEV₁ response relative to baseline following a reference COPD treatment, even if the reference COPD treatment has not been administered to the patient. As such, the patient would demonstrate a low FEV₁ response if the reference COPD treatment were administered to the patient. In some cases, the reference COPD treatment has previously been administered to the patient prior to treatment with the compound, and the patient has optionally been identified as demonstrating a low FEV₁ response.

The low FEV₁ response relative to baseline may comprise: an increase in FEV₁ AUC₀₋₁₂ₕ of less than 100 mL; and/or an increase in morning trough FEV₁ of less than 100 mL. For instance, the low FEV₁ response relative to baseline may comprise: an increase in FEV₁ AUC₀₋₁₂ₕ of less than about 60 mL; and/or an increase in morning trough FEV₁ of less than about 60 mL. In some instances, the low FEV₁ response relative to baseline comprises: an increase in FEV₁ AUC₀₋₁₂ₕ of less than 40 mL; and/or an increase in morning trough FEV₁ of less than 40 mL.

The low FEV₁ response relative to baseline may comprise the stated increase in FEV₁ AUC₀₋₁₂ₕ. The low FEV₁ response relative to baseline may comprise both the stated increase in FEV₁ AUC₀₋₁₂ₕ and the stated increase in morning trough FEV₁.

The low FEV₁ response relative to baseline may comprise an increase in FEV₁ AUC₀₋₁₂ₕ of less than 60 mL, less than 40 mL, less than 20 mL or less than 0 mL. The low FEV₁ response relative to baseline may comprise an increase in morning trough FEV₁ of less than 60 mL, less than 40 mL, less than 20 mL or less than 0 mL.

The patient may demonstrate an increase in FEV₁ AUC₀₋₁₂ₕ of at least −300 mL, at least −100 mL, at least −50 mL or at least 0 mL relative to baseline following a reference COPD treatment. The patient may demonstrate an increase in morning trough FEV₁ of at least −300 mL, at least −100 mL, at least −50 mL or at least 0 mL relative to baseline following a reference COPD treatment.

The patient may demonstrate an increase in FEV₁ AUC₀₋₁₂ₕ of from −300 mL to 100 mL, from −50 mL to 100 mL, from −300 mL to 60 mL, from −50 mL to 60 mL, from 0 mL to 60 mL, from −300 mL to 40 mL, from −100 mL to 40 mL, from 0 mL to 40 mL, or from −20 mL to 20 mL relative to baseline following a reference COPD treatment.

The patient may demonstrate an increase in morning trough FEV₁ of from −300 mL to 100 mL, from −50 mL to 100 mL, from −300 mL to 60 mL, from −50 mL to 60 mL, from 0 mL to 60 mL, from −300 mL to 40 mL, from −100 mL to 40 mL, from 0 mL to 40 mL, or from −20 mL to 20 mL relative to baseline following a reference COPD treatment.

Typically, the low FEV₁ response relative to baseline comprises an increase in morning trough FEV₁ of from −100 mL to 60 mL, for instance from 0 mL to 60 mL.

The baseline FEV₁ against which the changes in FEV₁ AUC₀₋₁₂ₕ and morning trough FEV₁ is typically as measured by the mean of two measurements taken before administration of the reference COPD treatment on the day of first dosing of the reference COPD treatment, i.e. 40 minutes and just prior to dosing.

FEV₁ AUC₀₋₁₂ₕ is defined as area under the curve over 12 hours of the FEV₁, divided by 12 hours. FEV₁ measurements are typically taken at 0.5, 1, 2, 4, 6 8 and 12 hours post dose with the reference COPD treatment.

Morning trough lung function can be measured by determining the FEV₁ of the patient shortly before the morning administration of the reference COPD treatment as part of a maintenance therapy with the reference COPD treatment. For instance, FEV₁ may be measured less than an hour before the morning administration of the reference COPD treatment. Morning trough FEV₁ may be the FEV₁ as measured between 11.5 and 12 hours following the prior evening dose.

The low FEV₁ response relative to baseline is typically as measured following treatment with the reference COPD treatment for at least 6 weeks. For instance, the low FEV₁ response relative to baseline may be as measured following treatment with the reference COPD treatment for at least 12 weeks. Thus, the patient may be determined to, or may be susceptible to, show a low FEV₁ response as defined herein following treatment for COPD for at least 6 weeks or at least 12 weeks.

Typically, the low FEV₁ response relative to baseline comprises an increase in FEV₁ AUC₀₋₁₂ₕ of from −200 mL to 40 mL, optionally following maintenance treatment with the reference COPD treatment for 12 weeks. The low FEV₁ response relative to baseline may comprise an increase in morning trough FEV₁ of from −100 mL to 30 mL, optionally following maintenance treatment with the reference COPD treatment for 12 weeks.

The reference COPD treatment typically comprises treatment with one or more bronchodilators. The reference COPD treatment may comprise treating the patient with the reference COPD treatment for from 2 to 12 weeks. The reference COPD treatment may comprise maintenance treatment with one or more bronchodilators.

The one or more bronchodilators of the reference COPD treatment are typically one or more of β₂-adrenergic agonists, muscarinic receptor antagonists and phosphodiesterase ¾ inhibitors. The muscarinic receptor antagonists are typically one or more of aclidinium, darotropium, tiotropium, glycopyrrolate and umeclidinium. The β₂-adrenergic agonists are typically one or more of salbutamol, salmeterol, formoterol, indacaterol, vilanterol, olodaterol, abediterol and carmoterol. For instance, the patient may be susceptible to resistance to improvements in lung function following treatment with one or more bronchodilators (other than ensifentrine), and the method may comprise choosing to initiate treatment with the compound which is ensifentrine or a pharmaceutically acceptable salt thereof.

Preferably, the one or more bronchodilators in the reference COPD treatment comprises ensifentrine or a pharmaceutically acceptable salt thereof. Thus, the patient may be resistant to improvements in lung function (or may be susceptible to resistance to improvements in lung function) as defined herein, following treatment with ensifentrine or a pharmaceutically acceptable salt thereof. As such, the method may comprise improving symptoms of COPD and/or QoL in a COPD patient with ensifentrine or a pharmaceutically acceptable salt thereof, wherein the patient shows a low FEV₁ response relative to baseline following treatment with ensifentrine or a pharmaceutically acceptable salt thereof. For example, the method of treating COPD may comprise continuing treatment with the compound (ensifentrine or a pharmaceutically acceptable salt thereof) in order to improve symptoms of COPD in the patient and/or the quality of life of the patient, after identifying that the patient demonstrates a low FEV₁ response relative to baseline following treatment with the compound.

The patient resistant to improvements in lung function may be a patient who demonstrates (or is susceptible to demonstrating) an increase in FEV₁ AUC₀₋₁₂ₕ of less than 100 mL relative to baseline and/or an increase in morning trough FEV₁ of less than 100 mL relative to baseline, each following daily administration of ensifentrine for a period of at least six weeks. For example, the patient resistant to improvements in lung function is a patient who demonstrates (or is susceptible to demonstrating) an increase in FEV₁ AUC₀₋₁₂ₕ of less than 40 mL relative to baseline and/or an increase in morning trough FEV₁ of less than 40 mL relative to baseline, each following daily administration of ensifentrine for a period of at least twelve weeks. In some cases, the patient resistant to improvements in lung function is a patient who demonstrates (or is susceptible to demonstrating) an increase in FEV₁ AUC₀₋₁₂ₕ of from −200 mL to 40 mL relative to baseline and/or an increase in morning trough FEV1 of from −100 mL to 30 mL relative to baseline, each following daily administration of ensifentrine for a period of at least twelve weeks. Daily administration of ensifentrine typically comprises twice daily administration of about 3 mg of ensifentrine, as described herein.

The patient may have been identified as being resistant to improvements in lung function. For instance, the patient may have been observed to demonstrate a low FEV₁ response relative to baseline following a reference COPD treatment as defined in herein. The method may therefore include a step of identifying the patient as being resistant to improvements in lung function and selecting the patient for treatment with the compound.

The Global Initiative for Chronic Obstructive Lung Disease (GOLD) classifies COPD into four distinct stages. These are mild COPD, moderate COPD, severe COPD or very severe COPD. The 2022 COPD report is published by the Global Initiative for Chronic Obstructive Disease, Inc, and that document is incorporated by reference in its entirety.

The above stages of COPD can be classified as set out below, where FEV₁ is forced expiratory volume in 1 second and FVC is forced vital capacity.
Mild COPD: FEV₁/FVC<0.7 and FEV₁≥80% predicted
Moderate COPD: FEV₁/FVC <0.7 and 50%≤FEV₁<80% predicted
Severe COPD: FEV₁/FVC <0.7 and 30%≤FEV₁<50% predicted
Very Severe COPD: FEV₁/FVC <0.7 and FEV₁<30% predicted

In each case the actual FEV₁ for the patient is compared with a predicted FEV₁ value based on factors such as age and height of the patient. These predicted values are readily available to the skilled person, for instance from the National Health and Nutrition Examination Survey III (Hankinson J L, Odencrantz J R, Fedan K B. Spirometry reference values from a sample of the general U.S. Population. Am J Respir Crit Care. 1999; 159:179-187). Examples of equations for calculating the predicted FEV₁ (in L) for a patient are as follows, where H is height (cm) and A is age (yrs):
Males: 0.0430H-0.0290A-2.490
Females: 0.0395H-0.025A-2.600

The FEV₁ and FVC used to determine the severity of COPD in a patient are measured by carrying out spirometry shortly after the administration of an adequate dose of at least one short-acting inhaled bronchodilator. Typically, measurement of FEV₁ and FVC for determining COPD disease severity is done between 15 and 30 minutes following administration of salbutamol (albuterol).

Typically, as used herein, FEV₁ and FVC are determined as set out in the article Standardisation of spirometry, Eur J 2005; 26; 319-338.

The patient may accordingly have been determined to have moderate COPD by measuring FEV₁/FVC <0.7 and 50%≤FEV1<80% predicted FEV₁ value, where FEV₁ is forced expiratory volume in 1 second and FVC is forced vital capacity as measured between 15 and 30 minutes after a dose of a bronchodilator, optionally wherein the bronchodilator is salbutamol. The determination of the patient's COPD severity may take place at least 1 day prior to the first administration of the compound.

The patient may have a baseline FEV₁ of at least 1500 mL, at least 2000 mL or at least 2500 mL. For instance, the patient may have a baseline FEV₁ of from 1500 mL to 5000 mL, or from 2000 mL to 4000 mL. The patient is typically suffering from moderate COPD, severe COPD or very severe COPD. For instance the patient may be suffering from moderate COPD. The patient may alternatively be suffering from severe or very severe COPD, for instance severe COPD.

The patient may be suffering from irreversible COPD. As such, the patient may be suffering from irreversible airflow obstruction.

A patient suffering from pulmonary emphysema may be predisposed to resistance to improvement in lung function following treatment. As such, the patient may be suffering from emphysema.

Treating COPD typically comprises improving the symptoms of COPD in the patient. Improving the symptoms of COPD typically comprises one or more of: reducing shortness of breath (dyspnea); reducing coughing; reducing mucus production; reducing chest discomfort; and increasing physical activity. For instance, improving the symptoms of COPD may comprise one or more of: reducing shortness of breath (dyspnea); reducing coughing; and reducing mucus production. Improving symptoms of COPD may comprise reducing the frequency and/or severity of COPD exacerbations in the patient.

Treating COPD may comprise improving the symptoms of COPD in the patient and improving the quality of life of the patient.

Treating COPD typically comprises: (i) improving the symptoms of COPD in the patient as determined by (a) a reduction of at least 2 in the Evaluating Respiratory Symptoms (E-RS) score of the patient or (b) an increase of at least 1 in the Transitional Dyspnea Index (TDI) score of the patient; and/or (ii) improving the quality of life of the patient as determined by a reduction of at least 4 in the St. George's Respiratory Questionnaire (SGRQ) score of the patient.

The E-RS score may be reduced by at least 3. The TDI score may be increased by at least 2. The SGRQ score may be reduced by at least 5 or at least 6.

Treating COPD may comprise reducing the E-RS score of the patient by at least 2 and/or increasing the TDI score of the patient by at least 1, relative to the scores within 24 hours before the patient is first treated with the compound. Treating COPD may comprise reducing the E-RS score as defined herein and increasing the TDI score as defined herein.

The changes in E-RS, TDI and SGRQ score are typically achieved at least 12 weeks after the patient is first treated with the compound. The changes in E-RS, TDI and SGRQ score may be achieved at least 24 weeks after the patient is first treated with the compound. For instance, treating COPD with the compound may comprise reducing the E-RS score of the patient by at least 2 and/or increasing the TDI score of the patient by at least 1 following at least 12 weeks of daily administration of the compound.

The patient may be male. The patient may be female. The patient may have an age of greater than or equal to 65 years. The patient may have an age of less than 65 years. The patient may be taking a background medication selected from one or more of a long-acting muscarinic antagonist (LAMA), a long-acting beta-agonist (LABA) and an inhaled corticosteroid (ICS).

The compound is ensifentrine or a pharmaceutically acceptable salt thereof. Pharmaceutically acceptable salts are well known to the skilled person. Typically, the compound is ensifentrine (i.e. ensifentrine free base).

The method comprises administering the compound to the patient, for instance by oral administration or by inhalation. The method typically comprises administering the compound to the patient by inhalation. A pharmaceutical composition comprising the compound and one or more pharmaceutically acceptable excipients or diluents is typically administered to the patient by inhalation, for instance by nebuliser, pressurised metered dose inhaler (pMDI) or dry powder inhaler (DPI).

Preferably, the method comprises administering the compound to the patient by inhalation from a nebuliser. Nebulisers aerosolise a liquid pharmaceutical composition into an aerosol that is inhaled into a patient's respiratory tract. Examples of nebulisers include a soft mist nebuliser, a vibrating mesh nebuliser, a jet nebuliser and an ultrasonic wave nebuliser. Suitable nebuliser devices include the Philips 1-neb™ (Philips), the Philips SideStream (Philips), the AeroNeb® (Philips), the Philips InnoSpire Go (Philips), the Pari LC Sprint (Pari GmbH), the AERxR™ Pulmonary Delivery System (Aradigm Corp) and the Pari LC Plus Reusable Nebuliser (Pari GmbH). The nebulizer may for instance be a PARI LC Sprint jet nebulizer with a PARI Vios® PRO Aerosol Delivery System PARI BOY® compressor. The compound may be inhaled via the nebuliser for from 1 to 15 minutes.

Typically, the method comprises administering the compound to the patient once, twice or three times per day, for instance twice or three times per day. The compound may be administered to the patient by inhalation once, twice or three times a day. Preferably the method comprises administering the compound to the patient by inhalation twice a day. The method may comprise administering a first dose of the compound in the morning (for instance within 3 hours following waking) and a second dose of the compound in the evening (for instance within 3 hours before bed). Typically, the morning and evening doses are administered from 10 to 14 hours apart, for instance about 12 hours apart.

The compound may be used in any suitable therapeutically effective amount. Typically, the daily dose of the compound is from 0.1 to 20 mg. Typically, the method comprises administering a total daily dose of the compound of from 0.5 to 10 mg. Preferably, the total daily dose of the compound (e.g. ensifentrine free base) is from 5 to 7 mg, for instance about 6 mg per day. As used herein, the term “about” may represent a variation of ±10% of the stated value. The total daily dose of the compound may be 6.0 mg.

Typically, the compound is administered twice a day in two separate doses which are the same or similar. For instance, the method may comprise administering the compound to the patient twice a day in a first dose of from 1 to 5 mg and a second dose of from 1 to 5 mg. Typically, the method may comprise administering the compound to the patient twice a day with a first dose of from 2 to 4 mg and a second dose of from 2 to 4 mg.

Preferably, the method comprises administering two doses of about 3 mg ensifentrine free base to the patient per day by inhalation. The method preferably comprises administering a dose of about 3 mg of the compound to the patient twice a day (3 mg BID) by inhalation. More preferably, the method comprises administering by nebuliser a dose of about 3 mg the compound to the patient twice a day. Each dose may be 3.0 mg free base ensifentrine administered by nebulizer.

The compound is typically used as a maintenance therapy. Typically, the method comprises administering the compound to the patient at least once per day for at least 8 weeks. The compound may be administered to the patient at least once per day for at least 16 weeks, preferably for at least 24 weeks. The compound may be administered daily to the patient for at least 1 year. The method may comprise administering the compound to the patient at least once every 24 hours, preferably at least twice every 24 hours, for at least 8 weeks, preferably for at least 16 weeks, more preferably for at least 24 weeks.

The compound is preferably administered as a suspension formulation, i.e. a suspension of particles comprising the compound in a diluent. The compound may alternatively be delivered as a dry powder, for instance a dry powder comprising particles comprising the compound and particles of a carrier such as lactose.

The method typically comprises administering an inhalable pharmaceutical composition comprising a suspension of particles of the compound in a diluent. The particles comprising the compound typically have a particle size distribution with a Dv50 of from 0.5 μm to 5.0 μm. The particles preferably have a Dv50 of from 1.0 μm to 2.0 μm.

Particle sizes are described herein by reference to the Dv50 value, which is the median particle size for a volume distribution. Thus, half the volume of the particles have diameters of less than the Dv50 value and half the volume of the particles have diameters of greater than the Dv50 value. This is a well-known manner in which to describe particle size distributions.

The technique used to measure the Dv50 values as stated herein is typically laser diffraction. For instance, the particle size distribution can be measured by laser diffraction using a Malvern Mastersizer 3000. Typically, the instrument parameters for the Malvern Mastersizer 3000 are as follows:
Non-spherical particle mode: Yes
Refractive index: 1.500
Absorption index: 0.500
Particle density: 1.00 g/cm³
Dispersant name: 0.02% Polysorbate 20 in water
Refractive index: 1.330
Stirrer; 1000 rpm
Sample measurement: 10 s
Obscuration: 10-20%

The particles comprising the compound typically comprise ensifentrine (i.e. ensifentrine free base). The particles may comprise at least 90 wt % ensifentrine free base relative to the total weight of the particles. The particles may comprise at least 99 wt % ensifentrine. The particles may consist of ensifentrine.

The concentration of particles comprising the compound in the inhalable pharmaceutical composition is typically from 0.1 to 5.0 mg/mL, preferably from 0.1 to 2.5 mg/mL, more preferably from 1.0 to 2.0 mg/mL.

In some embodiments, the concentration of ensifentrine or a pharmaceutically acceptable salt thereof (e.g., ensifentrine free base) in the inhalable pharmaceutical composition is at least 0.8 mg/mL. In some embodiments, the concentration of ensifentrine or a pharmaceutically acceptable salt thereof (e.g., ensifentrine free base) is at least about 0.9 mg/mL (e.g., 1 mg/mL, 1.1 mg/mL). In some embodiments, the concentration of ensifentrine or a pharmaceutically acceptable salt thereof (e.g., ensifentrine free base) is at most 1.5 mg/mL. the concentration of ensifentrine or a pharmaceutically acceptable salt thereof (e.g., ensifentrine free base) is at most 1.4 mg/mL (e.g., 1.3 mg/mL). In some embodiments, the concentration of ensifentrine or a pharmaceutically acceptable salt thereof (e.g., ensifentrine free base) is from about 1 mg/mL to about 1.4 mg/mL (e.g., 1.1 mg/mL to 1.3 mg/mL). In certain embodiments, the concentration of ensifentrine or a pharmaceutically acceptable salt thereof (e.g., ensifentrine free base) is about 1.2 mg/mL. In some embodiments, the concentration of ensifentrine free base in the inhalable pharmaceutical composition is (e.g., about) 1.2 mg/mL.

The inhalable pharmaceutical composition typically further comprises one or more tonicity adjusters, one or more buffers and one or more surfactants. The tonicity adjuster is typically sodium chloride.

In some embodiments, the tonicity adjuster (e.g., sodium chloride) is present in the inhalable pharmaceutical composition at a concentration of at least 1 mg/mL (e.g., 2 mg/mL, 3 mg/mL, 4 mg/mL, 5 mg/mL, 6 mg/mL, 7 mg/mL). In some embodiments, the tonicity adjuster (e.g., sodium chloride) is present in the inhalable pharmaceutical composition at a concentration of at most 15 mg/mL (e.g., 14 mg/mL, 13 mg/mL, 12 mg/mL, 11 mg/mL, 10 mg/mL, 9 mg/mL). In some embodiments, the tonicity adjuster (e.g., sodium chloride) is present in the pharmaceutical composition at a concentration of from about 1 mg/mL to about 15 mg/mL, about 3 mg/mL to about 10 mg/mL, about 4 mg/mL to about 10 mg/mL, about 5 mg/mL to about 11 mg/mL, about 5 mg/mL to about 9 mg/mL, or about 6 mg/mL to about 9 mg/mL. In some embodiments, the tonicity adjuster (e.g., sodium chloride) is present in the inhalable pharmaceutical composition at a concentration of about 7 mg/mL to about 10 mg/mL. In some embodiments, the tonicity adjuster (e.g., sodium chloride) is present in the inhalable pharmaceutical composition at a concentration of 8 mg/mL to 9 mg/mL. In certain embodiments, the tonicity adjuster (e.g., sodium chloride) is present in the inhalable pharmaceutical composition at a concentration of (e.g., about) 8.6 mg/mL.

Examples of buffers include a citrate buffer, a phosphate buffer, an acetate buffer, and a bicarbonate buffer. Preferably, the buffer is a phosphate buffer, for instance sodium dihydrogen phosphate dihydrate and/or disodium phosphate dihydrate.

In some embodiments, the inhalable pharmaceutical compositions provided herein further comprise one or more buffers. In some embodiments, the one or more buffers comprise one or more of a citrate buffer, a phosphate buffer, an acetate buffer, and/or a bicarbonate buffer. In certain embodiments, the buffer is a phosphate buffer. In some embodiments, the buffer comprises sodium dihydrogen phosphate dihydrate and/or disodium hydrogen phosphate dihydrate. In some embodiments, the buffer (e.g., sodium dihydrogen phosphate dihydrate and/or disodium hydrogen phosphate dihydrate) is present in the inhalable pharmaceutical composition at a concentration of at least 0.1 mg/mL (e.g., 0.2 mg/mL, 0.4 mg/mL, 0.8 mg/mL, 1 mg/mL, or 1.4 mg/mL). In some embodiments, the buffer (e.g., sodium dihydrogen phosphate dihydrate and/or disodium hydrogen phosphate dihydrate) is present in the inhalable pharmaceutical composition at a concentration of 4 mg/mL (e.g., 3.8 mg/mL, 3.3 mg/mL, 2.8 mg/mL, 2.5 mg/mL, or 2 mg/mL). In some embodiments, the buffer (e.g., sodium dihydrogen phosphate dihydrate and/or disodium hydrogen phosphate dihydrate) is present in the inhalable pharmaceutical composition at a concentration of about 1 mg/mL to about 2 mg/mL, about 1.2 mg/mL to about 1.8 mg/mL, or about 1.4 mg/mL to about 1.7 mg/mL. In some embodiments, the buffer (e.g., sodium dihydrogen phosphate dihydrate and/or disodium hydrogen phosphate dihydrate) is present in the inhalable pharmaceutical composition at a concentration of about 1.5 mg/mL to about 1.7 mg/mL.

In some embodiments, the inhalable pharmaceutical composition comprises sodium dihydrogen phosphate dihydrate at a concentration of at least 0.1 mg/mL (e.g., 0.2 mg/mL, 0.3 mg/mL, 0.4 mg/mL, 0.5 mg/mL, 0.6 mg/mL). In some embodiments, the inhalable pharmaceutical composition comprises sodium dihydrogen phosphate dihydrate at a concentration of at most 1.2 mg/mL (e.g., 1.1 mg/mL, 1.0 mg/mL, 0.9 mg/mL, 0.8 mg/mL). In some embodiments, the inhalable pharmaceutical composition comprises sodium dihydrogen phosphate dihydrate at a concentration of from about 0.5 mg/mL to about 0.9 mg/mL. In some embodiments, the inhalable pharmaceutical composition comprises sodium dihydrogen phosphate dihydrate at a concentration of about 0.7 mg/mL to about 0.8 mg/mL. In some embodiments, the inhalable pharmaceutical composition comprises sodium dihydrogen phosphate dihydrate at a concentration of about 0.7 mg/mL. In certain embodiments, the inhalable pharmaceutical composition comprises sodium dihydrogen phosphate dihydrate at a concentration of (e.g., about) 0.744 mg/mL.

In some embodiments, the inhalable pharmaceutical composition comprises disodium hydrogen phosphate dihydrate at a concentration of at least 0.1 mg/mL (e.g., 0.2 mg/mL, 0.3 mg/mL, 0.4 mg/mL, 0.5 mg/mL, 0.6 mg/mL, 0.7 mg/mL). In some embodiments, the inhalable pharmaceutical composition comprises disodium hydrogen phosphate dihydrate at a concentration of at most 1.2 mg/mL (e.g., 1.1 mg/mL, 1.0 mg/mL, 0.9 mg/mL). In some embodiments, the inhalable pharmaceutical composition comprises disodium hydrogen phosphate dihydrate at a concentration of from about 0.7 mg/mL to about 1 mg/mL. In some embodiments, the inhalable pharmaceutical composition comprises disodium hydrogen phosphate dihydrate at a concentration of about 0.8 mg/mL to about 0.9 mg/mL. In some embodiments, the inhalable pharmaceutical composition comprises disodium hydrogen phosphate dihydrate at a concentration of about 0.8 mg/mL. In some embodiments, the inhalable pharmaceutical composition comprises disodium hydrogen phosphate dihydrate at a concentration of about 0.9 mg/mL. In certain embodiments, the inhalable pharmaceutical composition comprises disodium hydrogen phosphate dihydrate at a concentration of (e.g., about) 0.853 mg/mL.

Examples of surfactants include lecithin, oleic acid, polyoxyethylene glycol alkyl ethers (for instance PEG 300, PEG 600, PEG 1000, Brij 30, Brij 35, Brij 56, Brij 76 and Brij 97), polypropylene glycol (for instance PPG 2000), glucoside alkyl ethers, polyoxyethylene glycol octylphenol ethers, polyoxyethylene glycol alkylphenol ethers, glycerol alkyl esters, polyoxyethylene glycol sorbitan alkyl esters (polysorbates, for instance polysorbate 20, polysorbate 40, polysorbate 60 and polysorbate 80), sorbitan alkyl esters (for instance sorbitan monolaurate (Span 20), sorbitan monooleate (Span 80) and sorbitan trioleate (Span 85)), cocamide MEA, cocamide DEA, dodecyldimethylamine oxide, block copolymers of polyethylene glycol and polypropylene glycol (poloxamers), block copolymers of polyethylene glycol and polypropylene oxide (for instance Pluronic surfactants), polyvinyl pyrrolidone K25, polyvinyl alcohol, oligolactic acid, sodium dioctyl sulfosuccinate and polyethoxylated tallow amine (POEA).

Preferably, the one or more surfactants comprise a polysorbate and/or a sorbitan alkyl ester. The one or more surfactants may for instance comprise polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate) or polysorbate 80 (polyoxyethylene (20) sorbitan monooleate). The one or more surfactants may for instance comprise sorbitan monolaurate (Span 20), sorbitan monooleate (Span 80) or sorbitan trioleate (Span 85). Preferably, the sterile liquid vehicle comprises polysorbate 20 and/or sorbitan monolaurate (Span 20).

In some embodiments, the inhalable pharmaceutical composition comprises the one or more surfactants (e.g., polysorbate 20 and/or sorbitan monolaurate) in a concentration of at least 0.01 mg/mL (e.g., 0.05 mg/mL, 0.1 mg/mL, 0.2 mg/mL, 0.3 mg/mL, 0.4 mg/mL). In some embodiments, the inhalable pharmaceutical composition comprises the one or more surfactants (e.g., polysorbate 20 and/or sorbitan monolaurate) in a concentration of at most 1 mg/mL (e.g., 0.9 mg/mL, 0.8 mg/mL, 0.7 mg/mL, 0.6 mg/mL). In some embodiments, the inhalable pharmaceutical composition comprises the one or more surfactants (e.g., polysorbate 20 and/or sorbitan monolaurate) in a concentration of from about 0.3 mg/mL to about 0.7 mg/mL. In some embodiments, the inhalable pharmaceutical composition comprises the one or more surfactants (e.g., polysorbate 20 and/or sorbitan monolaurate) at a concentration of from about 0.01 mg/mL to 2 mg/mL. In some embodiments, the inhalable pharmaceutical composition comprises the one or more surfactants (e.g., polysorbate 20 and/or sorbitan monolaurate) in a concentration of from about 0.4 mg/mL to about 0.6 mg/mL. In some embodiments, the inhalable pharmaceutical composition comprises the one or more surfactants (e.g., polysorbate 20 and/or sorbitan monolaurate) at a concentration of about 0.5 mg/mL. In some embodiments, the inhalable pharmaceutical composition comprises the one or more surfactants (e.g., polysorbate 20 and/or sorbitan monolaurate) at a concentration of about 0.6 mg/mL. In certain embodiments, the inhalable pharmaceutical composition comprises the one or more surfactants (e.g., polysorbate 20 and/or sorbitan monolaurate) in a concentration of (e.g., about) 0.55 mg/mL.

In some embodiments, the inhalable pharmaceutical composition comprises polysorbate 20 (e.g., Tween 20) in a concentration of at least 0.1 mg/mL (e.g., 0.2 mg/mL, 0.3 mg/mL, 0.4 mg/mL). In some embodiments, the inhalable pharmaceutical composition comprises polysorbate 20 (e.g., Tween 20) in a concentration of at most 1 mg/mL (e.g., 0.9 mg/mL, 0.8 mg/mL, 0.7 mg/mL, 0.6 mg/mL). In some embodiments, the inhalable pharmaceutical composition comprises polysorbate 20 (e.g., Tween 20) in a concentration of from about 0.3 mg/mL to about 0.7 mg/mL. In some embodiments, the inhalable pharmaceutical composition comprises polysorbate 20 (e.g., Tween 20) in a concentration of about 0.4 mg/mL to about 0.6 mg/mL. In certain embodiments, the inhalable pharmaceutical composition comprises polysorbate 20 (e.g., Tween 20) in a concentration of about 0.5 mg/mL.

In some embodiments, the inhalable pharmaceutical composition comprises sorbitan monolaurate (Span 20) in a concentration of at least 0.01 mg/mL (e.g., 0.02 mg/mL, 0.03 mg/mL, 0.04 mg/mL). In some embodiments, the inhalable pharmaceutical composition comprises sorbitan monolaurate (Span 20) in a concentration of at most 0.1 mg/mL (e.g., 0.09 mg/mL, 0.08 mg/mL, 0.07 mg/mL, 0.06 mg/mL). In some embodiments, the inhalable pharmaceutical composition comprises sorbitan monolaurate (Span 20) in a concentration of from about 0.03 mg/mL to about 0.07 mg/mL. In some embodiments, the inhalable pharmaceutical composition comprises sorbitan monolaurate (Span 20) in a concentration of from about 0.04 mg/mL to about 0.06 mg/mL. In certain embodiments, the inhalable pharmaceutical composition comprises sorbitan monolaurate (Span 20) in a concentration of (e.g., about) 0.05 mg/mL.

For instance, the method may comprise administering to the patient an inhalable liquid pharmaceutical composition comprising:
water;
particles consisting of ensifentrine free base at a concentration of from 0.1 to 20 mg/mL;
one or more tonicity adjusters at a total concentration of from 1.0 to 15 mg/mL;
one or more buffers at a total concentration of from 0.1 to 4 mg/mL; and
one or more surfactants at a total concentration of from 0.05 to 3 mg/mL.

The inhalable liquid pharmaceutical composition may comprise:
water;
particles consisting of ensifentrine free base at a concentration of from 0.5 to 6 mg/mL;
sodium chloride at a concentration of from 5 to 12 mg/mL;
sodium dihydrogen phosphate dihydrate at a concentration of from 0.3 to 2 mg/mL;
disodium phosphate dihydrate at a concentration of from 0.3 to 2 mg/mL;
polysorbate 20 at a concentration of from 0.1 to 1.5 mg/mL; and
sorbitan monolaurate at a concentration of from 0.01 to 0.5 mg/mL.

The compound may be used in combination with a second active agent. The compound may be administered separately or simultaneously with the second active agent. The patient may already be taking a second active agent as a background therapy for COPD. Alternatively, treatment with the second active agent may start at around the same time as treatment with the compound. The compound and the second active agent may be administered in a fixed combination.

The second active agent is typically selected from a muscarinic receptor antagonist, a beta-adrenergic receptor agonist and an inhaled corticosteroid. The compound may accordingly be used in combination with muscarinic receptor antagonist or a beta-adrenergic receptor agonist. The second active agent may be a long-acting muscarinic receptor antagonist (LAMA) or a long-acting beta-adrenergic receptor agonist (LABA).

Examples of LAMAs include aclidinium, darotropium, tiotropium, glycopyrrolate and umeclidinium. Examples of LABAs include salmeterol, formoterol, indacaterol, vilanterol, olodaterol, abediterol and carmoterol. Examples of inhaled corticosteroids include beclomethosone, budesonide, fluticasone propionate, ciclesonide, mometasone, fluticasone furoate and pharmaceutically acceptable salts thereof.

The compound may alternatively be used as a monotherapy. For instance, the compound may be for use in treating COPD as defined herein as the sole active agent. In some embodiments, the compound is not administered in combination with a muscarinic receptor antagonist or a beta-adrenergic receptor agonist.

The invention is described in more detail by the following Example.

### EXAMPLE

### Study Design

Clinical studies were conducted to determine the efficacy of ensifentrine in treating COPD compared with placebo. Ensifentrine was administered by nebuliser at a dose of 3 mg twice daily (BID) for 24 weeks. The studies were multi-centre, randomized, double-blind, parallel-group, placebo-controlled trials.

The study population included patients aged 40-80 years with moderate to severe COPD (FEV₁ 30%-70% p.n., FEV₁/forced vital capacity (FVC) ratio<0.7, with mMRC≥2). The randomization stratified (a) the use of stable background maintenance LAMA or LABA therapy use (approx. 50%. yes or no) and (b) cigarette smoking (current or former). Inhaled corticosteroid (ICS) maintenance therapy was permitted in up to 20% of patients under certain provisions.

The primary endpoint was change from baseline in average FEV₁ area under the curve AUC₀₋₁₂ₕ post-dose at week 12. Also assessed were COPD symptoms and quality of life (QoL) in patients showing lower responses in terms of lung function, and in particular patients with: (i) improvement in average FEV₁ AUC₀₋₁₂ₕ of less than 100 mL and (ii) improvement in morning trough FEV₁ of less than 100 mL, less than 60 mL or less than 40 mL.

### Methods

COPD severity is derived as follows: mild: 80%<=FEV₁, moderate: 50%<=FEV₁<80% predicted, severe: 30%<=FEV₁<50% predicted and very severe: FEV₁<30% predicted, post bronchodilator dose at Screening.

Baseline FEV₁ is the mean of the two measurements taken before study medication on the day of first dosing, i.e. 40 minutes and just prior to dosing, both pre-dose on day 1.

Average FEV₁ AUC₀₋₁₂ₕ is defined as area under the curve over 12 hours of the FEV₁, divided by 12 hours. FEV₁ measurements are typically taken at 0.5, 1, 2, 4, 6 8 and 12 hours post dose. Morning trough FEV₁ is defined as the FEV₁ assessed at 11.5 to 12 hours following the prior evening dose.

COPD exacerbations were defined as worsening of 2 or more major symptoms (dyspnea, sputum volume, and sputum purulence) or worsening of any 1 major symptom together with any 1 of the following minor symptoms: sore throat, colds, fever without other cause, and increased cough or wheeze for at least 2 consecutive days. COPD exacerbations were considered to be of moderate severity if treatment with systemic corticosteroids and/or antibiotics was required and were considered to be severe if hospitalization was required.

COPD symptoms were assessed using Evaluating Respiratory Symptoms (E-RS; EXACT-Respiratory Symptoms, The minimal clinically important difference (MCID) of the E-RS is a total score of −2 units. User Manual (Version 3.0), October 2014) and Transitional Dyspnea Index (TDI; Mahler et al, The MCID of the transition dyspnea index is a total score of one unit. COPD 2005, 2:99-103.) and QoL was assessed using the St. George's Respiratory Questionnaire (SGRQ; Jones, St. George's Respiratory Questionnaire: The MCID of the SGRQ is a total score of −4 units. COPD 2005, 2:75-79.).

In the results: CI=confidence interval; CSR=clinical study report; mITT=modified intent to treat.

### Formulation

The investigational product and placebo were provided in 2.5 mL unit dose format in an ampule and administered via a nebuliser. The formulation of the investigational product (ensifentrine suspension formulation) and placebo are shown in Table 1.

TABLE 1

| Constituent | Concentration (mg/mL) |
|---|---|
| Ensifentrine particles (RPL554) | 1.2 (for the active) or 0 (for Placebo) |
| Polysorbate 20 (Tween 20) | 0.50 |
| Sorbitan Monolaurate (Span 20) | 0.05 |
| Sodium Dihydrogen Phosphate Dihydrate | 0.744 |
| Disodium Hydrogen Phosphate Dihydrate | 0.853 |
| Sodium Chloride | 8.60 |
| Water | q.s. to 1 mL |

### Results

The primary endpoint of average FEV₁ (AUC)₀₋₁₂ₕ at Week 12 was met. All subgroups showed improvement in lung function with ensifentrine that was statistically significant.

Patients with improvement in average FEV₁ AUC₀₋₁₂ₕ less than 100 mL

Analyses of improvement in symptoms and QoL in patients who demonstrated a change from baseline in average FEV₁ AUC₀₋₁₂ₕ less than 100 mL at Week 12 confirmed that a higher proportion of symptom and QoL responders was achieved in patients receiving ensifentrine, and this effect was statistically or numerically greater than placebo at all weeks.

Improvements in symptoms and QoL in ensifentrine-treated patients were observed as early as the first assessment at Week 6, and were sustained over 24 weeks.

In this analysis of patients treated with ensifentrine who had an increase from baseline in average FEV₁ AUC₀₋₁₂ₕ less than 100 mL at Week 12, at least ⅔ saw a clinically important improvement in symptoms and nearly half saw a clinically important improvement in QoL over 24 weeks. These improvements are notable in the context of approximately 60% of patients in this subgroup being on background LAMA or LABA therapy.

Table 2 shows the analysis of the proportion of patients with improvement in average FEV1 AUC₀₋₁₂ₕ less than 100 mL and a clinically important response in E-RS (−2 Units) or TDI (+1 Unit) or SGRQ (−4 Units) (Pooled mITT).

A nominally statistically significant 37% reduction in moderate/severe exacerbation rate and risk (measured by time to first event) was also observed in this patient group, for patients treated with ensifentrine vs placebo, consistent with the full mITT analysis.

TABLE 2

| | | E-RS (−2 Unit) or TDI (+1 | | SGRQ (−4 Unit) | |
|---|---|---|---|---|---|
| | | Unit) Responders | | Responders | |
| Estimates from Logistic | | Ensifentrine | Placebo | Ensifentrine | Placebo |
| Regression | Statistic | (N = 975) | (N = 574) | (N = 975) | (N = 574) |

Week 6

| | | | | | |
|---|---|---|---|---|---|
| N Average FEV₁ AUC_{0-12 h} < | N | 631 | 478 | 623 | 473 |
| 100 mL at Week 12 | | | | | |
| % E-RS, TDI or SGRQ | % | 66.3 | 58.0 | 45.4 | 39.1 |
| Responders at Week 6 ^{a} | | | | | |
| Odds in favor of a | Odds | 1.69 | 1.19 | 1.10 | 0.84 |
| response | (95% CI) | (1.16, 2.46) | (0.82, 1.75) | (0.76, 1.60) | (0.57, 1.23) |
| Ensifentrine vs. Placebo | Odds | 1.42 | — | 1.32 | — |
| | Ratio | (1.09, 1.85) | | (1.01, 1.72) | |
| | (95% CI) | | | | |
| | p-value | 0.010 | — | 0.044 | — |

Week 12

| | | | | | |
|---|---|---|---|---|---|
| N Average FEV₁ AUC_{0-12 h} < | N | 631 | 478 | 623 | 473 |
| 100 mL at Week 12 | | | | | |
| % E-RS, TDI or SGRQ | % | 71.2 | 60.3 | 45.4 | 38.9 |
| Responders at Week 12 ^{a} | | | | | |
| Odds in favor of a | Odds | 2.69 | 1.65 | 0.92 | 0.70 |
| response | (95% CI) | (1.78, 4.08) | (1.09, 2.51) | (0.63, 1.34) | (0.48, 1.03) |
| Ensifentrine vs. Placebo | Odds | 1.63 | — | 1.31 | — |
| | Ratio | (1.22, 2.17) | | (0.99, 1.72) | |
| | (95% CI) | | | | |
| | p-value | <0.001 | — | 0.055 | 1 |

Week 24

| | | | | | |
|---|---|---|---|---|---|
| N Average FEV₁ AUC_{0-12 h} < | N | 631 | 478 | 623 | 473 |
| 100 mL at Week 12 | | | | | |
| % E-RS, TDI or SGRQ | % | 72.7 | 65.3 | 48.5 | 47.2 |
| Responders at Week 24 ^{a} | | | | | |
| Odds in favor of a | Odds | 2.91 | 2.10 | 0.95 | 0.91 |
| response | (95% CI) | (1.92, 4.40) | (1.36, 3.25) | (0.65, 1.40) | (0.61, 1.35) |
| Ensifentrine vs. Placebo | Odds | 1.38 | — | 1.04 | — |
| | Ratio | (1.02, 1.87) | | (0.79, 1.38) | |
| | (95% CI) | | | | |
| | p-value | 0.035 | — | 0.763 | — |

^{a} Multiple imputation range method.

Patients with improvement in morning trough FEV₁ less than 100 mL

Analyses of improvement in symptoms and QoL in patients who demonstrated a change from baseline in morning trough FEV₁ of less than 100 mL confirmed that a higher proportion of symptom and QoL responders was achieved in patients receiving ensifentrine over 24 weeks, and this effect was numerically greater than placebo at all weeks. Improvements in symptoms and QoL in ensifentrine treated patients were observed as early as the first assessment at Week 6, and were sustained over 24 weeks.

In this analysis of patients treated with ensifentrine who had an increase from baseline in morning trough FEV₁ less than 100 mL at Week 12, at least ⅔ saw a clinically important improvement in symptoms and nearly half saw a clinically important improvement in QoL over 24 weeks. These improvements are notable in the context of approximately 60% of patients in this subgroup being on background LAMA or LABA therapy.

Table 3 shows analysis of the proportion of patients with improvement in morning trough FEV₁ less than 100 mL and a clinically important response in E-RS (−2 Units) or TDI (+1 Unit) or SGRQ (−4 Units) (Pooled mITT).

A nominally statistically significant reduction of approximately 40% in moderate/severe exacerbation rate and risk (measured by time to first event) was also observed in patients treated with ensifentrine vs placebo, consistent with the full mITT analysis.

TABLE 3

| | | E-RS (−2 Unit) or TDI (+1 | | SGRQ (−4 Unit) | |
|---|---|---|---|---|---|
| | | Unit) Responders | | Responders | |
| Estimates from | | Ensifentrine | Placebo | Ensifentrine | Placebo |
| Logistic Regression | Statistic | (N = 975) | (N = 574) | (N = 975) | (N = 574) |

Week 6

| | | | | | |
|---|---|---|---|---|---|
| N Morning Trough | N | 690 | 469 | 681 | 464 |
| FEV₁ < 100 mL at | | | | | |

Week 6

| | | | | | |
|---|---|---|---|---|---|
| % E-RS, TDI or SGRQ | % | 66.1 | 57.7 | 45.0 | 38.9 |
| Responders ^{a} at Week | | | | | |
| 6 | | | | | |
| Odds in favor of a | Odds | 1.47 | 1.03 | 1.09 | 0.84 |
| response | (95% CI) | (1.01, 2.13) | (0.70, 1.51) | (0.76, 1.56) | (0.58, 1.22) |
| Ensifentrine vs. | Odds Ratio | 1.43 | — | 1.29 | — |
| Placebo | (95% CI) | (1.10, 1.86) | | (0.99, 1.67) | |
| | p-value | 0.008 | — | 0.058 | — |

Week 12

| | | | | | |
|---|---|---|---|---|---|
| N Morning Trough | N | 721 | 470 | 712 | 465 |
| FEV₁ < 100 mL at | | | | | |
| Week 12 | | | | | |
| % E-RS, TDI or SGRQ | % | 71.9 | 59.8 | 46.3 | 38.6 |
| Responders ^{a} at Week | | | | | |
| 12 | | | | | |
| Odds in favor of a | Odds | 2.96 | 1.71 | 0.80 | 0.60 |
| response | (95% CI) | (1.98, 4.43) | (1.14, 2.56) | (0.55, 1.16) | (0.41, 0.88) |
| Ensifentrine vs. | Odds Ratio | 1.73 | — | 1.34 | — |
| Placebo | (95% CI) | (1.31, 2.29) | | (1.03, 1.74) | |
| | p-value | <0.001 | — | 0.032 | — |

Week 24

| | | | | | |
|---|---|---|---|---|---|
| N Morning Trough | N | 741 | 457 | 730 | 452 |
| FEV₁ < 100 mL at | | | | | |
| Week 24 | | | | | |
| % E-RS, TDI or SGRQ | % | 72.0 | 64.5 | 48.1 | 47.4 |
| Responders ^{a} at Week | | | | | |
| 24 | | | | | |
| Odds in favor of a | Odds | 2.75 | 1.93 | 0.77 | 0.75 |
| response | (95% CI) | (1.86, 4.05) | (1.28, 2.91) | (0.53, 1.11) | (0.51, 1.10) |
| Ensifentrine vs. | Odds Ratio | 1.42 | — | 1.03 | — |
| | (95% CI) | (1.07, 1.90) | | (0.78, 1.34) | |
| Placebo | p-value | 0.017 | — | 0.855 | — |

^{a} Multiple imputation range method

Patients with improvement in morning trough FEV₁ less than 60 mL

An analogous analysis was conducted in the group of patients who had less than 60 mL increase in morning trough FEV₁. Given the large proportion of patients taking background LAMA or LABA therapy, this threshold was also assessed. Results of this analysis are shown in Table 4, and demonstrate results consistent with the analysis shown in Table 3, with the proportion of responders in symptoms or QoL favouring ensifentrine at all timepoints in both studies.

Table 4 shows analysis of the proportion of patients with improvement in morning trough FEV₁ less than 60 mL and a Clinically Important Response in E-RS (−2 Units) or TDI (+1 Unit) or SGRQ (−4 Units) (Pooled mITT).

TABLE 4

| | | TDI (+1 Unit) or E-RS (−2 | | SGRQ (−4 Unit) | |
|---|---|---|---|---|---|
| | | Unit) Responders | | Responders | |
| Estimates from | | Ensifentrine | Placebo | Ensifentrine | Placebo |
| Logistic Regression | Statistic | (N = 975) | (N = 574) | (N = 975) | (N = 574) |

Week 6

| | | | | | |
|---|---|---|---|---|---|
| N Morning Trough | N | 602 | 437 | 595 | 433 |
| FEV₁ < 60 mL at Week 6 | | | | | |
| % Responders ^{a} at | % | 65.3 | 56.9 | 43.4 | 37.8 |
| Week 6 | | | | | |
| Odds in favor of a | Odds | 1.35 | 0.96 | 0.98 | 0.79 |
| response | (95% CI) | (0.91, 2.01) | (0.64, 1.44) | (0.68, 1.42) | (0.54, 1.15) |
| Ensifentrine vs. | Odds | 1.42 | — | 1.25 | — |
| | Ratio | (1.07, 1.86) | | (0.95, 1.64) | |
| | (95% CI) | | | | |
| Placebo | p-value | 0.014 | — | 0.116 | — |

Week 12

| | | | | | |
|---|---|---|---|---|---|
| N Morning Trough | N | 648 | 426 | 639 | 421 |
| FEV₁ < 60 mL at Week | | | | | |
| 12 | | | | | |
| % Responders ^{a} at | % | 71.3 | 60.3 | 45.1 | 38.6 |
| Week 12 | | | | | |
| Odds in favor of a | Odds | 2.83 | 1.78 | 0.82 | 0.64 |
| response | (95% CI) | (1.80, 4.42) | (1.13, 2.80) | (0.54, 1.23) | (0.42, 0.97) |
| Ensifentrine vs. | Odds | 1.59 | — | 1.28 | — |
| Placebo | Ratio | (1.19, 2.13) | | (0.97, 1.69) | |
| | (95% CI) | | | | |
| | p-value | 0.002 | — | 0.084 | — |

Week 24

| | | | | | |
|---|---|---|---|---|---|
| N Morning Trough | N | 667 | 413 | 657 | 408 |
| FEV₁ < 60 mL at Week | | | | | |
| 24 | | | | | |
| % Responders ^{a} at | % | 71.7 | 65.2 | 48.2 | 46.4 |
| Week 24 | | | | | |
| Odds in favor of a | Odds | 2.74 | 2.02 | 0.79 | 0.73 |
| response | (95% CI) | (1.81, 4.15) | (1.30, 3.14) | (0.53, 1.17) | (0.48, 1.11) |
| Ensifentrine vs. | Odds | 1.36 | — | 1.07 | — |
| Placebo | Ratio | (1.00, 1.85) | | (0.81, 1.42) | |
| | (95% CI) | | | | |
| | p-value | 0.049 | — | 0.632 | — |

^{a} Multiple imputation range method

Patients with improvement in morning trough FEV₁ less than 40 mL

An analogous analysis was conducted in the group of patients who had less than 40 mL increase in morning trough FEV₁. Results of this analysis are shown in Table 5, and demonstrate results consistent with the analysis shown in Tables 3 and 4, with the proportion of responders in symptoms or QoL favouring ensifentrine at all timepoints in both studies.

Table 5 shows analysis of the proportion of patients with improvement in morning trough FEV₁ less than 40 mL and a Clinically Important Response in E-RS (−2 Units) orTDI (+1 Unit) or SGRQ (−4 Units) (Pooled mITT).

TABLE 5

| | E-RS (−2 Units) or TDI (+1 | | | |
|---|---|---|---|---|
| | Unit) Responders | | SGRQ (−4 Units) Responders | |
| | Ensifentrine | Placebo | Ensifentrine | Placebo |
| Statistic | (N = 975) | (N = 574) | (N = 975) | (N = 574) |

Week 6

| | | | | | |
|---|---|---|---|---|---|
| N Morning | N | 547 | 411 | 541 | 407 |
| Trough FEV₁ < | | | | | |
| 60 mL | | | | | |
| % responders | % | 67.1% | 57.9% | 42.6% | 37.4% |
| Odds in | Odds (95% | 1.453 | 0.990 | 0.903 | 0.717 |
| favour of | CI) | (0.9718, | (0.6592, | (0.6150, | (0.4850, |
| response | | 2.1734) | 1.4869) | 1.3272) | 1.0600) |
| Ensifentrine v | Odds Ratio | 1.468 | — | 1.260 | |
| placebo | (95% CI) | (1.1177, | | (0.9466, | |
| | | 1.9278) | | 1.6772) | |
| | p-value | 0.006 | — | 0.113 | |

Week 12

| | | | | | |
|---|---|---|---|---|---|
| N Morning | N | 596 | 404 | 588 | 400 |
| Trough FEV₁ < | | | | | |
| 60 mL | | | | | |
| % responders | % | 70.4% | 62.1% | 44.7% | 38.0% |
| Odds in | Odds (95% | 2.338 | 1.631 | 0.802 | 0.609 |
| favour of | CI) | (1.5519, | (1.0754, | (0.5305, | (0.3983, |
| response | | 3.5223) | 2.4752) | 1.2125) | 0.9326) |
| Ensifentrine v | Odds Ratio | 1.433 | — | 1.316 | — |
| placebo | (95% CI) | (1.0971, | | (0.9867, | |
| | | 1.8718) | | 1.7552) | |
| | p-value | 0.008 | — | 0.062 | — |

Week 24

| | | | | | |
|---|---|---|---|---|---|
| N Morning | N | 629 | 384 | 620 | 381 |
| Trough FEV₁ < | | | | | |
| 60 mL | | | | | |
| % responders | % | 73.0% | 66.5% | 48.7% | 45.2% |
| Odds in | Odds (95% | 3.238 | 2.363 | 0.797 | 0.700 |
| favour of | CI) | (2.2152, | (1.5806, | (0.5346, | (0.4591, |
| response | | 4.7322) | 3.5332) | 1.1887) | 1.0685) |
| Ensifentrine v | Odds Ratio | 1.370 | — | 1.138 | — |
| placebo | (95% CI) | (1.0542, | | (0.8505, | |
| | | 1.7807) | | 1.5235) | |
| | p-value | 0.019 | — | 0.384 | — |

### CONCLUSION

Altogether, these analyses demonstrate that even in subjects with change from baseline in FEV₁ AUC₀₋₁₂ₕ of <100 mL or with morning trough FEV₁<100 mL, <60 mL or <40 mL, meaningful positive effects on COPD symptoms, quality of life, and exacerbations were observed in a large proportion of patients treated with ensifentrine (symptom and QoL MCID achieved in approximately 70% and 50% of subjects, respectively).

The effect in the ensifentrine treatment group was consistently higher than that observed with placebo. These analyses further confirm ensifentrine efficacy and support the multi-faceted pharmacology, which cannot be characterized exclusively by its bronchodilatory effects.

### EMBODIMENTS

### Additional Embodiments Include

1. A compound for use in a method of treating chronic obstructive pulmonary disease (COPD) in a patient suffering from COPD, which compound is ensifentrine or a pharmaceutically acceptable salt thereof, wherein:
the patient is resistant to improvements in lung function; and
treating COPD comprises (i) improving the symptoms of COPD in the patient and/or (ii) improving the quality of life of the patient.

2. A compound for use according to embodiment 1, wherein the patient resistant to improvements in lung function is a patient who demonstrates a low FEV₁ response relative to baseline following a reference COPD treatment,
optionally wherein the low FEV₁ response relative to baseline comprises:
an increase in FEV₁ AUC₀₋₁₂ₕ of less than 100 mL; and/or
an increase in morning trough FEV₁ of less than 100 mL.

3. A compound for use according to embodiment 2, wherein the low FEV₁ response relative to baseline comprises an increase in morning trough FEV₁ of less than about 60 mL, optionally wherein the low FEV₁ response relative to baseline comprises an increase in morning trough FEV₁ of less than 40 mL.

4. A compound for use according to embodiment 2 or embodiment 3, wherein the low FEV₁ response relative to baseline comprises an increase in morning trough FEV₁ of from −100 mL to 60 mL.

5. A compound for use according to any one of embodiments 2 to 4, wherein the low FEV₁ response relative to baseline is as measured following treatment with the reference COPD treatment for at least 6 weeks, optionally following treatment with the reference COPD treatment for at least 12 weeks.

6. A compound for use according to embodiment 5, wherein the reference COPD treatment comprises treatment with one or more bronchodilators, optionally wherein the reference COPD treatment comprises maintenance treatment with one or more bronchodilators.

7. A compound for use according to embodiment 6, wherein the one or more bronchodilators are one or more β₂-adrenergic agonists, muscarinic receptor antagonists and phosphodiesterase ¾ inhibitors.

8. A compound for use according to embodiment 6 or embodiment 7, wherein the one or more bronchodilators comprises ensifentrine or a pharmaceutically acceptable salt thereof.

9. A compound for use according to any one of the preceding embodiments, wherein the patient resistant to improvements in lung function is a patient who demonstrates an increase in FEV₁ AUC₀₋₁₂ₕ of less than 100 mL relative to baseline and/or an increase in morning trough FEV₁ of less than 100 mL relative to baseline following daily administration of ensifentrine for a period of at least six weeks,
optionally wherein the patient resistant to improvements in lung function is a patient who demonstrates an increase in FEV₁ AUC₀₋₁₂ₕ of less than 40 mL relative to baseline and/or an increase in morning trough FEV₁ of less than 40 mL relative to baseline following daily administration of ensifentrine for a period of at least twelve weeks.

10. A compound for use according to any one of the preceding embodiments, wherein the patient has been identified as being resistant to improvements in lung function, optionally wherein the patient has been observed to demonstrates a low FEV₁ response relative to baseline following a reference COPD treatment as defined in any one of embodiments 2 to 9.

11. A compound for use according to any one of the preceding embodiments, wherein the patient has a baseline FEV₁ of at least 1500 mL, at least 2000 mL or at least 2500 mL.

12. A compound for use according to any one of the preceding embodiments, wherein the patient is suffering from moderate COPD, severe COPD or very severe COPD, preferably wherein the patient is suffering from moderate COPD.

13. A compound for use according to any one of the preceding embodiments, wherein the patient is suffering from irreversible COPD.

14. A compound for use according to any one of the preceding embodiments, wherein the patient is suffering from emphysema.

15. A compound for use according to any one of the preceding embodiments, wherein treating COPD comprises improving the symptoms of COPD in the patient.

16. A compound for use according to any one of the preceding embodiments, wherein improving the symptoms of COPD comprises one or more of: reducing shortness of breath (dyspnea); reducing coughing; reducing mucus production; reducing chest discomfort; and increasing physical activity.

17. A compound for use according to any one of the preceding embodiments, wherein treating COPD comprises:
(i) improving the symptoms of COPD in the patient as determined by a reduction of at least 2 in the Evaluating Respiratory Symptoms (E-RS) score of the patient or an increase of at least 1 in the Transitional Dyspnea Index (TDI) score of the patient; and/or
(ii) improving the quality of life of the patient as determined by a reduction of at least 4 in the St. George's Respiratory Questionnaire (SGRQ) score of the patient.

18. A compound for use according to any one of the preceding embodiments wherein treating COPD comprises reducing the E-RS score of the patient by at least 2 and/or increasing the TDI score of the patient by at least 1, relative to the scores when the patient is first treated with the compound.

19. A compound for use according to embodiment 17 or embodiment 18, wherein the changes in E-RS, TDI and SGRQ score are achieved at least 12 weeks after the patient is first treated with the compound, optionally at least 24 weeks after the patient is first treated with the compound.

20. A compound for use according to any one of the preceding embodiments, wherein the method comprises administering the compound to the patient by inhalation, optionally wherein the method comprises administering the compound to the patient by inhalation by nebuliser.

21. A compound for use according to any one of the preceding embodiments, wherein the compound is ensifentrine.

22. A compound for use according to any one of the preceding embodiments, wherein the method comprises administering the compound to the patient once, twice or three times per day.

23. A compound for use according to any one of the preceding embodiments, wherein the method comprises administering the compound to the patient twice a day.

24. A compound for use according to anyone of the preceding embodiments, wherein the method comprises administering the compound as a maintenance therapy.

25. A compound for use according to any one of the preceding embodiments, wherein the method comprises administering a total daily dose of the compound of from 0.5 to 10 mg, preferably from 5 to 7 mg.

26. A compound for use according to any one of the preceding embodiments, wherein the method comprises administering the compound to the patient twice a day in a first dose of from 2 to 4 mg and a second dose of from 2 to 4 mg.

27. A compound for use according to any one of the preceding embodiments, wherein the method comprises administering a dose of about 3 mg of the compound to the patient twice a day (3 mg BID), preferably wherein the method comprises administering by nebuliser a dose of about 3 mg of the compound to the patient twice a day.

30. A compound for use according to any one of the preceding embodiments, optionally wherein the method comprises administering the compound to the patient at least once per day for at least 8 weeks, preferably for at least 16 weeks, more preferably for at least 24 weeks.

31. A compound for use according to any one of the preceding embodiments, wherein the compound is used in combination with a muscarinic receptor antagonist, a beta-adrenergic receptor agonist or an inhaled corticosteroid.

32. A method for treating chronic obstructive pulmonary disease (COPD) in a patient suffering from COPD who is resistant to improvements in lung function comprising administering to the resistant patient a therapeutically effective amount of an inhalable pharmaceutical composition comprising ensifentrine or a pharmaceutically acceptable salt thereof in an amount effective for (i) improving the symptoms of COPD in the resistant patient and/or (ii) improving the quality of life of the resistant patient.

33. The method of embodiment 32, wherein the patient resistant to improvements in lung function is a patient who demonstrates a low FEV₁ response relative to baseline following a reference COPD treatment,
optionally wherein the low FEV₁ response relative to baseline comprises:
an increase in FEV₁ AUC₀₋₁₂ₕ of less than 100 mL; and/or
an increase in morning trough FEV₁ of less than 100 mL.

34. The method of embodiment 33, wherein the low FEV₁ response relative to baseline comprises an increase in morning trough FEV₁ of less than about 60 mL, optionally wherein the low FEV₁ response relative to baseline comprises an increase in morning trough FEV₁ of less than 40 mL.

35. The method of embodiment 33 or embodiment 34, wherein the low FEV₁ response relative to baseline comprises an increase in morning trough FEV₁ of from −100 mL to 60 mL.

36. The method of any one of embodiments 33-35, wherein the low FEV₁ response relative to baseline is as measured following treatment with the reference COPD treatment for at least 6 weeks, optionally following treatment with the reference COPD treatment for at least 12 weeks.

37. The method of embodiment 36, wherein the reference COPD treatment comprises treatment with one or more bronchodilators, optionally wherein the reference COPD treatment comprises maintenance treatment with one or more bronchodilators.

38. The method of embodiment 37, wherein the one or more bronchodilators are one or more β₂-adrenergic agonists, muscarinic receptor antagonists and phosphodiesterase ¾ inhibitors.

39. The method of embodiment 37 or embodiment 38, wherein the one or more bronchodilators comprises ensifentrine or a pharmaceutically acceptable salt thereof.

40. The method of any one of embodiments 32-39, wherein the patient resistant to improvements in lung function is a patient who demonstrates an increase in FEV₁ AUC₀₋₁₂ₕ of less than 100 mL relative to baseline and/or an increase in morning trough FEV₁ of less than 100 mL relative to baseline following daily administration of ensifentrine for a period of at least six weeks,
optionally wherein the patient resistant to improvements in lung function is a patient who demonstrates an increase in FEV₁ AUC₀₋₁₂ₕ of less than 40 mL relative to baseline and/or an increase in morning trough FEV₁ of less than 40 mL relative to baseline following daily administration of ensifentrine for a period of at least twelve weeks.

41. The method of any one of embodiments 32-40, wherein the patient has been identified as being resistant to improvements in lung function, optionally wherein the patient has been observed to demonstrates a low FEV₁ response relative to baseline following a reference COPD treatment as defined in any one of embodiments 2 to 9.

42. The method of any one of embodiments 32-41, wherein the patient has a baseline FEV₁ of at least 1500 mL, at least 2000 mL or at least 2500 mL.

43. The method of any one of embodiments 32-42, wherein the patient is suffering from moderate COPD, severe COPD or very severe COPD, preferably wherein the patient is suffering from moderate COPD.

44. The method of any one of embodiments 32-43, wherein the patient is suffering from irreversible COPD.

45. The method of any one of embodiments 32-44, wherein the patient is suffering from emphysema.

46. The method of any one of embodiments 32-45, wherein treating COPD comprises improving the symptoms of COPD in the patient.

47. The method of any one of embodiments 32-46, wherein improving the symptoms of COPD comprises one or more of: reducing shortness of breath (dyspnea); reducing coughing; reducing mucus production; reducing chest discomfort; and increasing physical activity.

48. The method of any one of embodiments 32-47, wherein treating COPD comprises:
(i) improving the symptoms of COPD in the patient as determined by a reduction of at least 2 in the Evaluating Respiratory Symptoms (E-RS) score of the patient or an increase of at least 1 in the Transitional Dyspnea Index (TDI) score of the patient; and/or
(ii) improving the quality of life of the patient as determined by a reduction of at least 4 in the St. George's Respiratory Questionnaire (SGRQ) score of the patient.

49. The method of any one of embodiments 32-48, wherein treating COPD comprises reducing the E-RS score of the patient by at least 2 and/or increasing the TDI score of the patient by at least 1, relative to the scores when the patient is first treated with the inhalable pharmaceutical composition.

50. The method of embodiment 48 or embodiment 49, wherein the changes in E-RS, TDI and SGRQ score are achieved at least 12 weeks after the patient is first treated with the inhalable pharmaceutical composition, optionally at least 24 weeks after the patient is first treated with the inhalable pharmaceutical composition.

51. The method of any one of embodiments 32-50, wherein the method comprises administering the inhalable pharmaceutical composition to the patient by inhalation, optionally wherein the method comprises administering the inhalable pharmaceutical composition to the patient by inhalation by nebuliser.

52. The method of any one of embodiments 32-51, wherein the compound is ensifentrine.

53. The method of any one of embodiments 32-52, wherein the method comprises administering the inhalable pharmaceutical composition to the patient once, twice or three times per day.

54. The method of any one of embodiments 32-53, wherein the method comprises administering the inhalable pharmaceutical composition to the patient twice a day.

55. The method of any one of embodiments 32-54, wherein the method comprises administering the inhalable pharmaceutical composition as a maintenance therapy.

56. The method of any one of embodiments 32-55, wherein the method comprises administering a total daily dose of the compound of from 0.5 to 10 mg, preferably from 5 to 7 mg.

57. The method of any one of embodiments 32-56, wherein the method comprises administering the compound to the patient twice a day in a first dose of from 2 to 4 mg and a second dose of from 2 to 4 mg.

58. The method of any one of embodiments 32-57, wherein the method comprises administering a dose of about 3 mg of the compound to the patient twice a day (3 mg BID), preferably wherein the method comprises administering by nebuliser a dose of about 3 mg of the compound to the patient twice a day.

59. The method of any one of embodiments 32-58, optionally wherein the method comprises administering the inhalable pharmaceutical composition to the patient at least once per day for at least 8 weeks, preferably for at least 16 weeks, more preferably for at least 24 weeks.

60. The method of any one of embodiments 32-59, wherein the inhalable pharmaceutical composition is used in combination with a muscarinic receptor antagonist, a beta-adrenergic receptor agonist or an inhaled corticosteroid.

61. The method of any one of embodiments 32-60, wherein the inhalable pharmaceutical composition comprises a suspension of ensifentrine particles comprising the ensifentrine or a pharmaceutically acceptable salt thereof in a diluent.

62. The method of any one of embodiments 32-61, wherein the inhalable pharmaceutical composition comprises:
a. ensifentrine particles;
b. a buffer;
c. one or more surfactants; and
d. a tonicity adjuster,
wherein a total weight of the ensifentrine or the pharmaceutically acceptable salt thereof in the liquid pharmaceutical composition is of from about 2.7 mg to about 3.3 mg.

63. The method of any one of embodiments 32-61, wherein the inhalable pharmaceutical composition comprises:
a. ensifentrine particles at a concentration of from about 1 mg/mL to about 1.4 mg/mL;
b. a buffer at a concentration of from about 1 mg/mL to about 2 mg/mL;
c. one or more surfactants at a total concentration of from about 0.01 mg/mL to about 0.8 mg/mL; and
d. a tonicity adjuster at a concentration of from about 5 mg/mL to about 10 mg/mL.

64. The method of anyone of embodiments 32-61, wherein the inhalable pharmaceutical composition comprises:
a. ensifentrine particles at a concentration of from about 1 mg/mL to about 1.4 mg/mL;
b. polysorbate 20 (Tween 20) at a concentration of from about 0.3 mg/mL to about 0.7 mg/mL;
c. sorbitan monolaurate (Span 20) at a concentration of from 0 mg/mL to about 0.1 mg/mL;
d. sodium dihydrogen phosphate dihydrate at a concentration of from about 0.5 mg/mL to about 1 mg/mL;
e. disodium hydrogen phosphate dihydrate at a concentration of from about 0.5 mg/mL to about 1 mg/mL; and
f. sodium chloride at a concentration of from about 5 mg/mL to about 10 mg/mL.

65. The method of anyone of embodiments 32-61, wherein the inhalable pharmaceutical composition comprises:
a. 1.2 mg/mL ensifentrine particles;
b. 0.5 mg/mL polysorbate 20;
c. 0.05 mg/mL sorbitan monolaurate;
d. 0.744 mg/mL sodium dihydrogen phosphate dihydrate;
e. 0.853 mg/mL disodium hydrogen phosphate dihydrate; and
f. 8.6 mg/mL sodium chloride; and
g. water.

66. The method of anyone of embodiments 32-61, wherein the inhalable pharmaceutical composition is a suspension comprising:
a. 1.4 mg/mL ensifentrine particles;
b. 0.55 mg/ml polysorbate 20;
c. 0.744 mg/ml sodium dihydrogen phosphate dihydrate;
d. 0.853 mg/ml disodium hydrogen phosphate dihydrate;
e. 8.6 mg/ml sodium chloride; and
f. water.

67. The method of any one of embodiments 32-61, wherein the inhalable pharmaceutical composition is a suspension comprising:
a. 1.3 mg/mL ensifentrine particles;
b. 0.55 mg/ml sorbitan monolaurate;
c. 0.744 mg/ml sodium dihydrogen phosphate dihydrate;
d. 0.853 mg/ml disodium hydrogen phosphate dihydrate;
e. 8.6 mg/ml sodium chloride; and
f. water.

68. The method of any one of embodiments 32-61, wherein the inhalable pharmaceutical composition is a suspension comprising:
a. 1.3 mg/mL ensifentrine particles;
b. 0.5 mg/ml polysorbate 20;
c. 0.05 mg/ml sorbitan monolaurate;
d. 1.1 mg/ml sodium dihydrogen phosphate dihydrate;
e. 0.9 mg/ml disodium hydrogen phosphate dihydrate;
f. 13 mg/ml sodium chloride; and
g. water.

69. The method of any one of embodiments 32-61, wherein the inhalable pharmaceutical composition is a suspension comprising:
a. 1.2 mg/mL ensifentrine particles;
b. 0.4 mg/ml polysorbate 20;
c. 0.10 mg/ml sorbitan monolaurate;
d. 0.744 mg/ml sodium dihydrogen phosphate dihydrate;
e. 0.853 mg/ml disodium hydrogen phosphate dihydrate;
f. 8.6 mg/ml sodium chloride; and
g. water.

70. The method of any one of embodiments 32-61, wherein the inhalable pharmaceutical composition is a suspension comprising:
a. 1.4 mg/mL ensifentrine particles;
b. 0.4 mg/ml polysorbate 20;
c. 0.04 mg/ml sorbitan monolaurate;
d. 1.2 mg/ml sodium dihydrogen phosphate dihydrate;
e. 8.6 mg/ml sodium chloride; and
f. water.

71. The method of any one of embodiments 32-61, wherein the inhalable pharmaceutical composition comprises:
a. the ensifentrine particles at a concentration of from 0.8 to 1.4 mg/mL;
b. one or more surfactants at a total concentration of from 0.3 to 0.7 mg/mL;
c. one or more buffers at a total concentration of from 1.0 to 2.0 mg/ml;
d. one or more tonicity adjusters at a concentration of from 1.0 to 15.0 mg/ml and
e. water.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

1. A method for treating chronic obstructive pulmonary disease (COPD) in a patient suffering from COPD who is resistant to improvements in lung function comprising administering to the resistant patient a therapeutically effective amount of an inhalable pharmaceutical composition comprising ensifentrine or a pharmaceutically acceptable salt thereof in an amount effective for (i) improving the symptoms of COPD in the resistant patient and/or (ii) improving the quality of life of the resistant patient.

2. The method of claim 1, wherein the patient resistant to improvements in lung function is a patient who demonstrates a low FEV₁ response relative to baseline following a reference COPD treatment.

3. The method of claim 2, wherein the low FEV₁ response relative to baseline comprises an increase in morning trough FEV₁ of less than about 60 mL.

4. The method of claim 2, wherein the low FEV₁ response relative to baseline is as measured following treatment with the reference COPD treatment for at least 6 weeks.

5. The method of claim 2, wherein the reference COPD treatment comprises treatment with one or more bronchodilators.

6. The method of claim 1, wherein the patient resistant to improvements in lung function is a patient who demonstrates an increase in FEV₁ AUC₀₋₁₂ₕ of less than 100 mL relative to baseline and/or an increase in morning trough FEV₁ of less than 100 mL relative to baseline following daily administration of ensifentrine for a period of at least six weeks.

7. The method of claim 1, wherein the patient has a baseline FEV₁ of at least 1500 mL, at least 2000 mL or at least 2500 mL.

8. The method of claim 1, wherein the patient is suffering from moderate COPD, severe COPD or very severe COPD.

9. The method of claim 1, wherein the patient is suffering from irreversible COPD.

10. The method of claim 1, wherein treating COPD comprises improving the symptoms of COPD in the patient.

11. The method of claim 1, wherein treating COPD comprises:
(i) improving the symptoms of COPD in the patient as determined by a reduction of at least 2 in the Evaluating Respiratory Symptoms (E-RS) score of the patient or an increase of at least 1 in the Transitional Dyspnea Index (TDI) score of the patient; or
(ii) improving the quality of life of the patient as determined by a reduction of at least 4 in the St. George's Respiratory Questionnaire (SGRQ) score of the patient.

12. The method of claim 11, wherein the changes in E-RS, TDI or SGRQ score are achieved at least 12 weeks after the patient is first treated with the inhalable pharmaceutical composition.

13. The method of claim 1, wherein the method comprises administering the inhalable pharmaceutical composition to the patient by inhalation.

14. The method of claim 1, wherein the method comprises administering the inhalable pharmaceutical composition to the patient once, twice or three times per day.

15. The method of claim 1, wherein the method comprises administering the inhalable pharmaceutical composition as a maintenance therapy.

16. The method of claim 1, wherein the method comprises administering a total daily dose of the compound of from 0.5 to 10 mg.

17. The method of claim 1, wherein the method comprises administering the compound to the patient twice a day in a first dose of from 2 to 4 mg and a second dose of from 2 to 4 mg.

18. The method of claim 1, wherein the method comprises administering a dose of about 3 mg of the compound to the patient twice a day (3 mg BID).

19. The method of claim 1, wherein the method comprises administering the inhalable pharmaceutical composition to the patient at least once per day for at least 8 weeks.

20. The method of claim 1, wherein the compound is used in combination with a muscarinic receptor antagonist, a beta-adrenergic receptor agonist or an inhaled corticosteroid.

21. The method of claim 1, wherein the inhalable pharmaceutical composition comprises a suspension of ensifentrine particles comprising the ensifentrine or a pharmaceutically acceptable salt thereof in a diluent.

22. The method of claim 21, wherein the inhalable pharmaceutical composition comprises:
a. ensifentrine particles;
b. a buffer;
c. one or more surfactants; and
d. a tonicity adjuster,
wherein a total weight of the ensifentrine or the pharmaceutically acceptable salt thereof in the liquid pharmaceutical composition is of from about 2.7 mg to about 3.3 mg.

23. The method of claim 21, wherein the inhalable pharmaceutical composition comprises:
a. ensifentrine particles at a concentration of from about 1 mg/mL to about 1.4 mg/mL;
b. a buffer at a concentration of from about 1 mg/mL to about 2 mg/mL;
c. one or more surfactants at a total concentration of from about 0.01 mg/mL to about 0.8 mg/mL; and
d. a tonicity adjuster at a concentration of from about 5 mg/mL to about 10 mg/mL.

24. The method of claim 21, wherein the inhalable pharmaceutical composition comprises:
a. ensifentrine particles at a concentration of from about 1 mg/mL to about 1.4 mg/mL;
b. polysorbate 20 (Tween 20) at a concentration of from about 0.3 mg/mL to about 0.7 mg/mL;
c. sorbitan monolaurate (Span 20) at a concentration of from 0 mg/mL to about 0.1 mg/mL;
d. sodium dihydrogen phosphate dihydrate at a concentration of from about 0.5 mg/mL to about 1 mg/mL;
e. disodium hydrogen phosphate dihydrate at a concentration of from about 0.5 mg/mL to about 1 mg/mL; and
f. sodium chloride at a concentration of from about 5 mg/mL to about 10 mg/mL.

25. The method of claim 21, wherein the inhalable pharmaceutical composition comprises:
a. 1.2 mg/mL ensifentrine particles;
b. 0.5 mg/mL polysorbate 20;
c. 0.05 mg/mL sorbitan monolaurate;
d. 0.744 mg/mL sodium dihydrogen phosphate dihydrate;
e. 0.853 mg/mL disodium hydrogen phosphate dihydrate;
f. sodium chloride at a concentration of 8.6 mg/mL; and
g. water.

26. The method of claim 21, wherein the inhalable pharmaceutical composition is a suspension comprising:
a. 1.4 mg/mL ensifentrine particles;
b. 0.55 mg/ml polysorbate 20;
c. 0.744 mg/ml sodium dihydrogen phosphate dihydrate;
d. 0.853 mg/ml disodium hydrogen phosphate dihydrate;
e. 8.6 mg/ml sodium chloride; and
f. water.

27. The method of claim 21, wherein the inhalable pharmaceutical composition is a suspension comprising:
a. 1.3 mg/mL ensifentrine particles;
b. 0.55 mg/ml sorbitan monolaurate;
c. 0.744 mg/ml sodium dihydrogen phosphate dihydrate;
d. 0.853 mg/ml disodium hydrogen phosphate dihydrate;
e. 8.6 mg/ml sodium chloride; and
f. water.

28. The method of claim 21, wherein the inhalable pharmaceutical composition is a suspension comprising:
a. 1.3 mg/mL ensifentrine particles;
b. 0.5 mg/ml polysorbate 20;
c. 0.05 mg/ml sorbitan monolaurate;
d. 1.1 mg/ml sodium dihydrogen phosphate dihydrate;
e. 0.9 mg/ml disodium hydrogen phosphate dihydrate;
f. 13 mg/ml sodium chloride; and
g. water.

29. The method of claim 21, wherein the inhalable pharmaceutical composition is a suspension comprising:
a. 1.2 mg/mL ensifentrine particles;
b. 0.4 mg/ml polysorbate 20;
c. 0.10 mg/ml sorbitan monolaurate;
d. 0.744 mg/ml sodium dihydrogen phosphate dihydrate;
e. 0.853 mg/ml disodium hydrogen phosphate dihydrate;
f. 8.6 mg/ml sodium chloride; and
g. water.

30. The method of claim 21, wherein the inhalable pharmaceutical composition is a suspension comprising:
a. 1.4 mg/mL ensifentrine particles;
b. 0.4 mg/ml polysorbate 20;
c. 0.04 mg/ml sorbitan monolaurate;
d. 1.2 mg/ml sodium dihydrogen phosphate dihydrate;
e. 8.6 mg/ml sodium chloride; and
f. water.

31. The method of claim 21, wherein the inhalable pharmaceutical composition comprises:
a. the ensifentrine particles at a concentration of from 0.8 to 1.4 mg/mL;
b. one or more surfactants at a total concentration of from 0.3 to 0.7 mg/mL;
c. one or more buffers at a total concentration of from 1.0 to 2.0 mg/ml;
d. one or more tonicity adjusters at a concentration of from 1.0 to 15.0 mg/ml and
e. water.
